(19) United States
(12) Patent Application Publication
(10) Pub. No.: US 2011/0060406 A1
(43) Pub. Date: Mar. 10, 2011

(54) HEART VALVE

(76) Inventors: Aparna Thirumalai Anandampillai, Chennai (IN); Vijayan Thirumalai Anandampillai, Chennai (IN)

(21) Appl. No.: 12/312,453

(22) PCT Filed: Nov. 27, 2007
(86) PCT No.: PCT/IN2007/000553

§ 371 Date: May 12, 2009

(30) Foreign Application Priority Data
Nov. 29, 2006 (IN) 2209/CHE/2006

(51) Int. Cl. A61F 2/24

(52) U.S. Cl. 623/2.13

(57) ABSTRACT

The new valve has a ring (1) with grooved (10) upper stents (2), three for trileaflet valves. The ring has a threaded mesh (6) stretching across stents. An outer sewing ring (4) placed on a gutter (3) on the outer surface of the ring fixes to native valve. The stent has grooves (10) to prevent sliding of mesh. The valve is sterilized and packed safely. The pericardium is cut by a levered cutter (7) with a handle, a middle fulcrum and the cutting cusp shaping (8) surfaces cutting desired size & shape cusps (5). The cusps are sutured to the mesh on indicated coloured threads. The valve is tested for leaks. The valve holder (9) holds the cusp mounted valve in suturing to native valve ring. The sewing ring of the valve is sutured to native valve annulus and the valve holder is removed. The valve opens with a big central lumen and closes with no leak.

### TECHNICAL FIELD

This invention in general relates to medical devices for heart, specifically a heart valve using patients own live pericardium for valve creation with a central natural flow. This valve is created in operating theater itself in minutes.

### STATE OF ART

Heart valves allow unidirectional flow of blood. Natural valves have a central, non-turbulent flow and coapt to close flow with no leak. Valves are destroyed by various diseases and replaced by artificial valves. Artificial valves have a metal ring with plastic (carbon) plates or balls that move to open, for flow and close. Artificial valves have a turbulent noncentral flow that promotes clotting, needing anticlot drugs with frequent blood tests for monitoring. Tissue artificial valves (bioprosthesis) are made of pig or cattle pericardium that is shaped as leaflets supported on stents which deteriorate fast needing resurgery. Recently stem cells grown on a plastic manifold are tried but it may take years to prove. The problems of mesh deterioration will take more years to prove. Cadaveric valves have a risk of viral disease as HIV, matching and is in short supply.

Defects in the Existing Artificial Valves (Plastic)

1. Need life time anticlot drugs and close monitoring of coagulation.

2. These are unphysiological with obstructed flow, have higher gradients to open the valves and some leak in closing.

3. They are prone to infection. Hence they have complications, costly monitoring and are not ideal valves.

Defects in the Existing Bioprosthetic Artificial Valves:

1. Early wear and calcification as the leaflets do not withstand long years of stress.

2. They need mild anticlot drugs and antigen destruction.

3. They need calcium preventing treatment.

4. Not good for kids and young adults

5. These are dead tissues like leather, have no regeneration or repair capacity and susceptible for infections.

Natural valves grow with age, withstand stresses of variable heart rates and can repair infections. The valve has an annulus ring with 2 or 3 leaflets arising from inside of annulus with a semicircular free margin closing below the edge. Leaflets open without gradient for a large central opening and close with no leak. The energy conversion to blood pumping is good with no turbulence, protecting blood cells & proteins! In bioprosthetic and human preserved pericardial valves the leaflets are fixed to a stent on each side and the annulus ring on pheriphry. This leads to a stress on the free margin and top surface of the dead leaflets. Stiff preservation chemicals kill the live cells loosing self repair. Altered collagen calcifies, looses pliability and leaks needing a reoperation. There is danger of zoonotic virus, from porcine & bovine specimen. Cadaveric valves need HIV and other screening, is a dead tissue and in short supply. Other artificial valves use nonlife pyrolitic carbon discs, balls with obstructed turbulent flow needing a large anti clot drugs. These have high gradient.

An extensive search has been carried out using the Internet and related patent specifications were studied for live human pericardial valve leaflets with a central flow with good leaflet support. The new invention has a live untreated same person pericardium with a different structure of leaflet support, always closing below the edges as in normal valves, leading to a pliable (not stiff) low gradient live valve that can fight infections. Live pericardial patches are used to close heart holes in children that last a life term.

U.S. Pat. No. 4,470,157 Jack W. Love is an autogenous valve which utilized mating stents to clamp the tissue between the stents but often prolapsed resulting in valve leakage. Another problem is the tendency for the tissue between the stents to slip, due to irregularities in tissue used in the valve interfering with the clamping force generated by the stents.

U.S. Pat. No. 4,687,483 describes a valve with number of pins and studs extending from an inner frame to holes and slots in an outer frame, securing the pins with securing washers, and sewing tissue or cloth frame coverings together. Because of the large number of pins and studs involved, it is a slow assembly of an autogenous tissue valve in the operating room. U.S. Pat. No. 4,192,020 is a valve utilizing an adhesive such as polyurethane dissolved in tetrahydrofuran to secure fabric to wire frames. Toxic adhesive is not suitable for affixing tissue, especially viable human tissue, to a valve.

U.S. Pat. No. 4,501,030 describes a complex valve which utilizes a significant number of sutures to assemble the valve. The stents in all these valves have a problem of cusp sliding and prolapsing. It is an object of the present invention to provide a rapid assembly flexible tissue valve that decreases sewing time and accuracy. Further object is to provide a valve which achieves proper alignment and prevents movement of the tissue during valve assembly to prevent prolapse. It is a further object to provide a method for assembling such a valve which is standardized and reproducible, and which can easily be learned by non-surgeon.

Ideal valve must have a smooth pliable soft closing and opening with no gradient & leaks. There must be a central nonturbulent flow with low load on the heart muscles. There are no ideal valves of the natural type for humans.

Further the invention is addressed to the process of using the new valve which is unique in design and construction, working and use with lower gradient.

Further objects of the invention will be clear from the ensuring description.

### SUMMARY OF THE INVENTION

The valve comprises of a plastic or plastic with metal ring. There are three stents supports projecting on one side. A mesh of pliable stiff threads wound on the ring and on the stent forms the cusp support. The three stents have three cusps (leaflets) support. The ring has an outer sewing ring for fixation to valve ring of native human heart. The pericardium is cut in a scaffold cutter and assembled on the thread mesh cusps, the pericardium is sutured to mesh by sutures on to the ring too in periphery. The completed valve is held in a holder and fixed to the native valve ring through sutures on the sewing ring. The holder is released. The three cusps form a live leaflet that opens and closes as natural valve.

### STATEMENT OF DRAWINGS

These and other objects and features of the invention will become more apparent upon perusal of following description taken in conjunction with accompanying drawings wherein:

FIG. 1 shows the various components of the novel valve.

FIG. 2 shows the exploded view of the novel inhaler with cusp cutter.

FIG. 3 shows the mechanism of closing of valve (a) and opening (b).

FIG. 4 shows details of conventional bioprosthesis valve

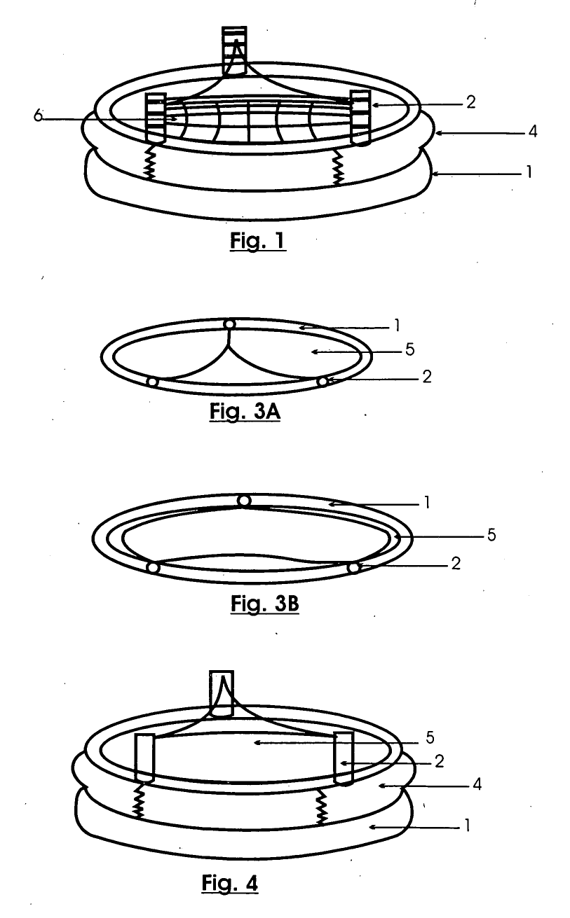

### DESCRIPTION OF PREFERRED EMBODIMENTS

The following specification describes salient features of invention, the method of construction, the method of use and the advantages of the novel invention.

This invention relates generally to a rapid assembly, live and flexible cusped tissue heart valve with easy sewing and more specifically, to a valve which can be assembled from prefabricated kit by a non-surgeon in the limited time available in an operating room, then securely hold valve assembly, and sew to the native valve ring.

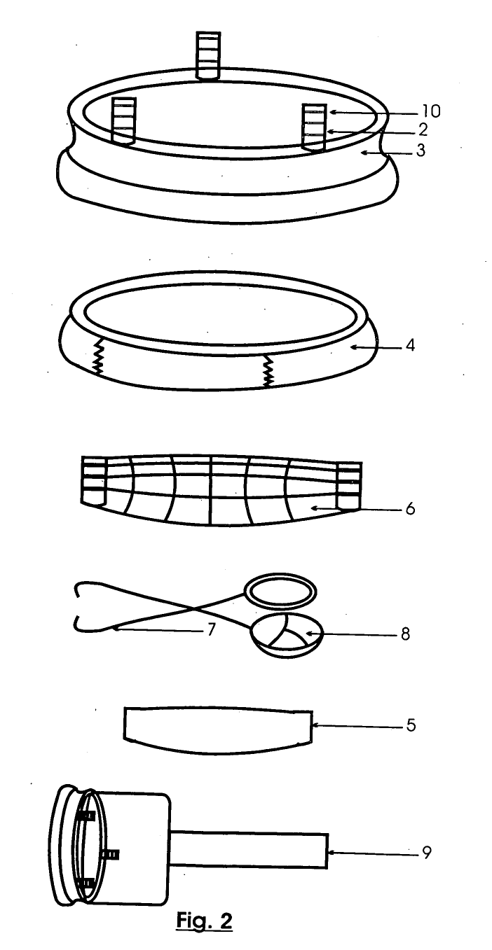

The conventional existing bioprosthesis valve FIG. 4 are made in factories by trained persons and consists of a plastic/metal ring (1) with a upper stent (2) at the top in which stiff leathery treated animal leaflet cusp (5) is anchored. The ring body has at the outer side has a sewing ring (4). The cusps are made of treated bovine/porcine pericardium or valve cusps (5). The cusps are sutured to the ring and the stent, the stiff dead cusps opens easily and close with stresses on the edge and top cusp surface. There is no regeneration in these killed valve cusp and soon they wear out. The valve is assembled in a factory, sterilized sealed and sold. The valve is held in a holder. The sutures are placed on the sewing ring and fixed. The device has not been improved for decades.

The novel valve has a plastic ring (1) with an upper three short stent supports (2) for a three cusp valve. Threads (6) tied between the stents and the ring to the above cross thread on the stents (2) form a mesh. These cross linked mesh support the pericardial cusp. The ring has on its outside surface a depression or gutter (3) for a sewing ring (4) FIG. 2, which fixes the valve to native valve annulus. Cut pericardial leaflets are sutured to the mesh forming the cusp (5). The cusps are tied to the ring, the stent and free margin of mesh. The mesh helps to hold the cusp with no stress on the pericardium in closing or opening! The thread mesh absorbs the top surface pressures for a longer life of valve. The Pericardium is cut by a cutter (7) which has a handle, a fulcrum and the cutting surface (8). The cutting surface is faced for leaflet shape and gives three cusps. The cutter is made of stainless steel for sterilization. The cutter is opened and pericardium is placed between cutting surface. Handle is closed to cut the cusps. The cutter is opened to remove three cusps. The cusps (5) FIG. 2 are placed on the valve ring mesh and sutured by fine interrupted needled sutures on the edge, stents and the ring. The three cusps are sutured and this completed valve is placed in a holder (9). Some threads are coloured for fast easy suturing by any worker. A Holder clamps the completed valve ring and the valve is sutured to native valve ring by sutures on sewing ring (4).

The valve cusps are better destressed by the new stent suturing and the mesh support. The threads are non stretching biocompatible polyester/prolene or the like. The stent has ringed grooves (10) surface for easy knotting and slide prevention after manufacture. Some threads are different coloured for easy pericardial cusp suturing to thread mesh by any worker. The mesh is designed for each size by a computed stress analysis of pressure points of closed valve. The valve with thread mesh is packed and sterilised by known methods, ethylene oxide or radiation or the like.

In the operating theater, patients chest opened, pericardium is exposed. Pericardium cutter is opened and introduced over pericardium and closed to cut three cusps. The heart is opened, the diseased valve is exposed, if to be replaced is cut to dissect the valve ring. The valve is sized. The needed size valve is opened under sterility. The cups are transferred to the valve thread mesh and sutured on indicated thread. About 6 inches of stitching is only needed, made easy by coloured threads. The valve is tested for opening, closing and leaks and transferred to holder for suturing on to native valve ring as in other valve replacements. The pericardium can be sutured to mesh by illiterates too at the coloured sites in few minutes of simple knotting. The cusp is sutured to ring, stents and free margin forming a pliable live valve.

The valve opens as natural valve by a pressure from below FIG. 3b with a large central nonturbulent flow and closes by passive rebound without leak FIG. 3a. as seen from top

The device can be modified. For the aortic valve the stents (2) may be bent slightly inwards, the ring may be triflanged with stents on the flanges. For mitral valve the stents are conical, narrower at the top at the ring slightly widening down with grooves, to prevent the slide of the cusps as this valve faces down—opposite direction in aortic the stents are conical with the narrow end on the ring. These grooves prevent slipping of the mesh. Two pericardial leaflets for each mesh surfaces may be used. Radio opaque stents and rings will show on x-rays for check ups if needed. The ring may be made of bio compatible plastics and or metals like titanium or a combination of both.

The valve is living, more pliable than fixed treated bioprosthesis with no fear of viral diseases. Peritoneum may be used instead of pericardium. The ring may have two concentric components—one for sewing ring and another fitting in this with stents and cusps. The inner valve ring may be placed and tightened in outer ring saving on time of valve creation. One surgeon may implant the ring, the other the valve cusp saving time and twisted in place later. The ring with the stent and its gutter are made as a single cast of plastic or metal without stress on stent ring joint. Any biocompatible smooth surface, non clotting sheets can be used as leaflets if the pericardium, peritoneum is diseased by cancer, infections. Instead of threads mesh, pliable thin nitinol or such wires may be used. The sewing ring may be made of Dacron, polyester cloth or any fibrin depositing material.

It will be apparent to those skilled in the art that modifications to the invention described herein can readily be made without departing from the spirit of the invention. Protection is sought for all the subject matter described herein including any such modifications.

### ADVANTAGES OF THE NEW INVENTION

1. Live self pericardium is free with no outside viral infections.

2. Mesh surface protects the cusps and decreases the stretching forces.

3. Live pericardium has better defense against infections.

4. No anti clot tests and drugs are needed.

5. The flow is central with no turbulence, no leaks.

6. The gradient and work to open the leaflets is lower as pericardium floats than artificial disks.

7. Mesh keeps the elasticity with easy opening and closure unlike bio prosthetic stiff leaflets.

8. Other serosa as peritoneum can be used.

9. The cutter is an easy first order lever for easy cutting.

We claim:

1) An improved heart valve for use instead of the existing dead stiff animal tissue biological valves wherein the improvement comprises of live self pericardial tissue fixed on a supporting mesh forming the pliable cusp of the valve proper, the said valve ring (1) having two or more upper short stents (2), the said stents (2) anchor and support interlocked thread meshes (6) in the shape of cusps between the said stents and the said valve ring, the said valve ring (1) having on its outer surface a gutter (3) at its middle with a sewing ring (4), the said pericardium cut as a leaflet cusps (5) of needed size by the pericardial cutter (7), the said cut pericardial cusp is fixed to said valve thread mesh (6) by sutures, the said completed valve is held in a valve holder (9) and sutured to the native valve of the heart.

2) A live improved pericardial valve as claimed in claim 1, wherein the said cusp mesh (6) is made of a series of strong but pliable, tissue compatible thread wound between the said stents (2) and the ring with interlinks in the form and size of cusp to fix and support the moving pericardial cut cusp, the said threads differently coloured at pericardial cusp suturing sites for fast easy suturing, the said mesh supports the said pericardial cusps to form an integrated valve leaflet for correct closing and opening without stress.

3) A live improved pericardial valve as claimed in claim 1, wherein the said ring has as many needed stents (2) as the cusps, the said stent receiving the ends of the said mesh cusp to arrest the fall of the leaflets in closure and support the said leaflets against closure pressures.

4) A live improved pericardial valve as claimed in claim 1, wherein, the said ring (1) with stents is made of biocompatible non-stick rigid plastic or metal or a composite, the said ring with the said stents forms the frame work of the said valve, the said ring has on its outer surface a gutter (3) for a sewing ring.

5) A live improved pericardial valve as claimed in claim 1, wherein the said sewing ring (4) is formed of Dacron/polyester, collagen, fibrin depositing cloth and shaped as a close fitting ring on the said ring gutter (3).

6) A live improved pericardial valve as claimed in claim 1 wherein the said stents (2) has multiple ringed grooves (10) for the said cusp mesh thread fixation, the said stent grooves preventing cusp sliding in fixation and use

7) A live improved pericardial valve as claimed in claim 1 wherein the said valve ring (1) is made as two concentric rings, the outer with sewing ring for native valve and inner valve proper with stented mesh for saving time, easy fixing and removal in surgery.

8) A live improved pericardial valve as claimed in claim 1 wherein the stent (2) of the ring in aortic valve is an upper tapering with anti sliding grooves prevents the cusp from sliding on the said stent.

9) A live improved pericardial valve as claimed in claim 1 wherein the stent (2) of the ring in mitral valve widens down from the ring with anti sliding grooves (10) prevents the cusp from sliding on the said stent.

10) A live improved pericardial valve as claimed in claim 1, wherein the said valve with the mesh in place is sterilized, packed in sterile safe containers.
